(19) United States
(12) Patent Application Publication
(10) Pub. No.: US 2024/0189372 A1
(43) Pub. Date: Jun. 13, 2024

(54) ARTHROSPIRA FOR USE IN THE TREATMENT OF DISEASES

(71) Applicant: BRANDENBURGISCHE TECHNSCH UNIVERSITÄT COTTBUS-SENFTENBERG, Senftenberg (DE)

(72) Inventors: Friedrich JUNG, Dresden (DE); Jan-Heiner KÜPPER, Kleinkoscken (DE)

(21) Appl. No.: 18/022,760

(22) PCT Filed: Aug. 24, 2021
(86) PCT No.: PCT/EP2021/073430

§ 371 Date: Feb. 23, 2023

(30) Foreign Application Priority Data
Aug. 24, 2020 (EP) 20192425.5

(51) Int. Cl. A61K 35/748; A61K 38/16; A61L 27/28; A61L 27/54; A61L 31/08; A61L 31/16; A61P 9/14

(52) U.S. Cl. CPC A61K 35/748; A61K 38/164; A61L 27/28; A61L 27/54; A61L 31/08; A61L 31/16; A61P 9/14; A61L 2300/30; A61L 2300/416

(57) ABSTRACT

The present invention refers to beneficial effects of an extract of Arthrospira or phycobiliproteins with respect to endothelial cells and related diseases.

Hereto, in a first aspect, the present invention relates to a pharmaceutical composition, in particular a coating comprising an extract of Arthrospira or phycobiliproteins as an active ingredient for implantable devices due to said beneficial effects.

Hereto, in a second aspect, the present invention relates to a pharmaceutical preparation/composition comprising an extract of Arthrospira or phycobiliproteins as an active ingredient for therapeutic and prophylactic treatment or prevention of neo-intimal hyperplasia, restenosis, thrombosis, embolism, bacterial infections, preferably bloodstream infections, primary bacteremia and sepsis due to said beneficial effects.

The present invention refers to beneficial effects of an extract of Arthrospira or phycobiliproteins with respect to endothelial cells and related diseases.

Hereto, in a first aspect, the present invention relates to a pharmaceutical composition, in particular a coating comprising an extract of Arthrospira or phycobiliproteins as an active ingredient for implantable devices due to said beneficial effects.

Hereto, in a second aspect, the present invention relates to a pharmaceutical preparation/composition comprising an extract of Arthrospira or phycobiliproteins as an active ingredient for therapeutic and prophylactic treatment or prevention of neo-intimal hyperplasia, restenosis, thrombosis, embolism, bacterial infections, preferably bloodstream infections, primary bacteremia and sepsis due to said beneficial effects.

Arthrospira is a genus of free-floating filamentous multicellular, photosynthesizing cyanobacterium characterized by cylindrical, multicellular trichomes in an open left-hand helix. A dietary supplement is made from A. platensis and A. maxima, known as Spirulina. Arthrospira, in particular Spirulina is rich in proteins, vitamins, essential amino acids, minerals and essential fatty acids [1]. Beyond its use as forage with known effects on flesh, egg and plumage color, milk yield and fertility, it has been found to have additional pharmacological properties. Many preclinical and a few clinical studies suggest several therapeutic effects ranging from reduction of cholesterol to enhancement of the immune system, an increase in intestinal lactobacilli, a detoxification of heavy metals and drugs and antioxidant, anti-inflammatory properties [1-4].

The excessive generation of intracellular reactive oxygen species (ROS) has been implicated in the pathogenesis of many cardiovascular diseases, including atherosclerosis, hypertension, heart failure and the associated endothelial dysfunction [5]. Dartsch reported a dose-dependent inactivation of free superoxide radicals (anti-oxidant effect) as well as an anti-inflammatory effect characterized by a dose-dependent reduction of the metabolic activity of neutrophils and a dose-dependent inactivation of superoxide radicals generated during an oxidative burst [5]. In addition, Chu et al. could show that the aqueous extract of Spirulina had a protective effect against apoptotic cell death due to free radicals in fibroblasts [6]. This antioxidant potential has been attributed mainly to phycobiliproteins prepared from Spirulina by aqueous extraction [7, 8]. A purified peptide from Arthrospira, in particular Spirulina inhibited the Angiotensin II induced production of intracellular reactive oxygen species (ROS) in a human endothelial cell line [9]. Moreover, the changes in cell morphology correlated with intracellular ROS production and were recovered by treatment with the purified peptide.

These findings were confirmed in an animal study in rats, in which the protective effect of Spirulina against 4-nitroquinoline-1-oxide (4NQO) induced hepato- and nephron-toxicity was explored. The 4NQO administration increased the oxidative stress with a concomitant decline in the levels of non-enzymic [reduced glutathione (GSH)] and enzymic antioxidants [(Superoxide dismutase (SOD), Catalase (CAT), Glutathione peroxidase (GPx), and Glutathione-S-transferase (GST)] in both liver and kidney and resulted in increased levels of hepatic and renal markers [Alanine Transaminase (ALT), Aspartate Transaminase (AST), Lactate Dehydrogenase (LDH), urea, creatinine and uric acid] in the serum of experimental animals. The oral pretreatment with aqueous extract of Spirulina prevented those 4NQO-induced changes in the levels of hepatic and kidney enzymes in the serum of experimental rats. It counteracted the 4NQO induced lipid peroxidation and maintained the hepatic and kidney antioxidant defense system at near normal in both liver and kidney [10].

However, the state of the art does not refer to the beneficial effects of an extract of Arthrospira, in particular Spirulina Arthrospira and/or phycobiliproteins with respect to endothelial cells.

The endothelial cell (EC) monolayer is a crucial component of the normal vascular wall, providing an interface between the bloodstream and the surrounding tissue of the blood vessel wall. ECs are also involved in physiological events including angiogenesis, inflammation and the prevention of thrombosis. It is evident that each phase of the vascular response to injury is influenced or may be controlled by the endothelium. Thus, disturbances of endothelial functions by toxic endogenous or exogenous substances such as produced by certain bacteria can have dramatic physiological consequences. Moreover, ECs have been encouraged to grow on the surface of stents by local delivery of vascular endothelial growth factor (VEGF), an endothelial cell mitogen, after implantation of a stent.

Therefore, the inventors have conducted a study in order to analyze whether an extract of Arthrospira, in particular Spirulina and/or phycobiliproteins may influence the endothelial cell monolayer formation in tissue culture plates and whether Arthrospira, in particular Spirulina could affect the toxic influence of bacterial toxins like lipopolysaccharides (LPS) on primary human venous endothelial cells (HUVEC).

Surprisingly, the inventors have found that an extract of Arthrospira, in particular Spirulina and/or phycobiliproteins have a protective and curative effect with respect to endothelial cells and the endothelial cell monolayer formation. Such an advantageous effect of an extract of Spirulina and/or phycobiliproteins are well supported by the presented examples and figures.

Accordingly, the present invention is directed in one aspect to the use of an extract of Arthrospira, in particular Spirulina in the manufacture of a medicament or a dietary supplement for use in treating or preventing a disease or condition benefiting from said protective and curative effect with respect to endothelial cells and the endothelial cell monolayer formation.

Moreover, there is a large need to provide novel applications for an extract of Arthrospira, in particular Spirulina in order to exploit its healthy and curative capacity. An extract of Spirulina is an advantageously safe and natural composition without any known side effects and disadvantages.

Hence, an extract of Arthrospira, in particular Spirulina is useful for a clinical or therapeutically interaction with endothelial cells and related diseases, in particular using implantable devices for the prophylaxis and treatment of such diseases.

The term “Arthrospira” in accordance with the present invention shall mean a genus of free-floating filamentous multicellular, photosynthesizing cyanobacterium characterized by cylindrical, multicellular trichomes in an open left-hand helix. A dietary supplement is preferably made from biomass of A. platensis and/or A. maxima, known as Spirulina. Spirulina is commonly used and a commercial name of a variety of similar cyanobacterial species belonging to the genus Arthrospira. The genus Arthrospira includes but is not limited to the following species: A. platensis, A. maxima, A. fusiformis, A. indica, A. innermongoliensis, A. jenneri, A. massartii and A. erdosensis. In accordance with the present invention the following species are preferred, namely A. platensis, A. maxima and A. fusiformis or commercially available Spirulina.

An extract of Arthrospira, in particular Spirulina may be preferably obtained by aqueous extraction [6], cf. example 2. Such aqueous extract can be commercially obtained from e.g., Sigma Aldrich, Munich, Germany.

In a further preferred embodiment of the invention the extract of Arthrospira, in particular Spirulina is enriched with bioactive preferably own ingredients or compounds of the native biomass, e.g. by means of concentration, fractionation or addition. In particular, such bioactive compounds are selected from the group of secondary plant substances, preferably photosynthetically active pigments, phycobiliproteins, phycocyanin, xanthophyll, chlorophyll, beta-carotene, echinenone, xanthine, fatty acids, linolenic acid (ALA), linoleic acid, stearidonic acid (SDA), eicosapentaenoic acid (EPA), docosahexaenoic acid (DHA), arachidonic acid (AA), oligosaccharides, and polyphenols.

In a further preferred embodiment, the enriched extract of Arthrospira, in particular Spirulina comprises more than 15 w/w phycobiliproteins, preferably, more than 30 w/w phycobiliproteins, or more than 45 w/w phycobiliproteins, or more than 60 w/w phycobiliproteins, or more than 75 w/w phycobiliproteins, or more than 90 w/w phycobiliproteins up to 99 w/w phycobiliproteins. The weight ratio (w/w) is calculated with reference to the dry weight. In a preferred embodiment the extract, incl. phycobiliproteins is a solid substance, in particular a powder.

Phycobiliproteins can be enriched by preparing a Spirulina extract as described in Example 2 followed by generation of dry mass by means of lyophilization or other known methods.

In order to measure enrichment, the purity of C-phycocyanin can be evaluated using the absorbance ratio of A620/A280, wherein a purity of 0.7 is considered as food grade, 3.9 as reactive grade and greater than 4.0 as analytical grade for phycocyanin [11].

Kumar et al describe a simple protocol for purifying phycocyanin from Spirulina platensis by using ammonium sulphate precipitation, followed by a single step chromatography using DEAE-Cellulose-11 and acetate buffer. Precipitation with 65% ammonium sulphate results in 80% recovery of phycocyanin with a purity of 1.5 (A620/A280). Through chromatography an 80% recovery of phycocyanin with a purity of 4.5 (A620/A280) can be achieved [12].

Such enriched extract of Arthrospira, in particular Spirulina are obtainable or obtained by using columns and collecting fractions. Moreover, preferably water or buffer, in particular isotonic buffer, is used as extraction agent. If needed, phycobiliproteins may be enriched to almost 100% w/w providing a pure form or pure substance.

Phycobiliproteins is an oligomeric protein consisting of equal numbers of alpha and beta subunits with a molecular weight of about 18 and 21 kDa, respectively. The alpha/beta-pairs mostly build the pigment as a trimer or hexamer. Both alpha and beta subunits have a bilin chromophore, which contains linear tetrapyrrole rings that are attached to the cysteine amino acid of the apoprotein by thioether linkages [13].

Particularly, phycocyanin belongs to the family of phycobiliproteins which are located in the supramolecular phycobilisomes on the external surface of the thylakoid membrane of cyanobacteria and act as major photosynthetic accessory pigments. They include phycoerythrin (PE), phycocyanin (C-PC), and allophycocyanin (A-PC), with C-phycocyanin in relatively high quantity [14]. Phycocyanin has a simply detectable characteristic light blue color, absorbing orange and red light, particularly near 620 nm, and emits fluorescence at about 650 nm.

Hence, phycobiliproteins, in particular phycocyanin in a pure form or provided in an almost pure substance is also suitable for all disclosed embodiments according to the invention.

In accordance with the invention, the term “phycobiliproteins” shall mean and encompass the following compounds selected from the group of phycocyanin, C-phycocyanin, allophycocyanin (syn.: A-phycocyanin), R-phycocyanin and phycoerythrin.

Accordingly, the present invention is directed to the use of phycobiliproteins in the manufacture of a medicament or a dietary supplement for use in treating or preventing a disease or condition benefiting from said protective and curative effect with respect to endothelial cells and the endothelial cell monolayer formation.

Implantable devices include, for example, stents, stent-grafts, synthetic bypass grafts, embolic filters, occluder systems, detachable coils, pacemaker and defibrillator leads, plates, screws, spinal cages, dental implants, ventricular assist devices, artificial hearts, artificial heart valves, annuloplasty devices, artificial joints, and implantable sensors. Frequently, implanted medical apparatus must be designed to be sufficiently biocompatible to the host body.

Otherwise, the body will manifest a rejection of the implant by way of a thrombotic, inflammatory or other deleterious response. Moreover, restenosis involves recoil and shrinkage of the vessel. Subsequently, recoil and shrinkage of the vessel are followed by proliferation of medial smooth muscle cells in response to injury of the vessel. In response to blood vessel injury, smooth muscle cells in the tunica media and fibroblasts of the adventitial layer undergo phenotypic change which results in the secretion of metalloproteases into the surrounding matrix, luminal migration, proliferation and protein secretion. Various other inflammatory factors are also released into the injured area including thromboxane A2, platelet derived growth factor (PDGF) and fibroblast growth factor (FGF).

Such implantable devices, therefore, are designed or fabricated from materials possessing surface properties that minimize bodily response at the tissue device interface and an injury caused to the tissue may occur during the implantation procedure (e.g. percutaneous transluminal coronary balloon angioplasty (PTCA), etc.), in particular the endothelial cell monolayer may be injured in the blood vessels.

Endothelial cell growth factors and environmental conditions in situ are therefore essential in modulating endothelial cell adherence, growth and differentiation at the site of blood vessel injury. Accordingly, with respect to restenosis and other blood vessel diseases, there is a need for the development of new methods and compositions for coating medical devices, which would promote and accelerate the formation of a functional endothelium on the surface of implanted devices so that a confluent EC monolayer is formed on the target blood vessel segment or grafted lumen and preventing or treating neo-intimal hyperplasia, or preventing or treating restenosis or preventing or treating thrombosis or preventing or treating embolism.

The present invention provides a medical device for implanting into the lumen of a blood vessel or an organ with a lumen as disclosed in the claims. The medical device comprises a coating comprising an extract of Arthrospira, in particular Spirulina.

Hence, the present invention refers to a pharmaceutical composition comprising an extract of Arthrospira, in particular Spirulina and/or phycobiliproteins for use in the prevention or treatment of a blood vessel disease selected from the group of neo-intimal hyperplasia, restenosis, thrombosis or embolism.

In particular, the present invention refers to a pharmaceutical composition comprising an extract of Arthrospira, in particular Spirulina and/or phycobiliproteins for use in the regeneration, in particular re-endothelization and/or acceleration of forming endothelial cells, in particular endothelial cell monolayer, wherein for example endothelial cells, in particular endothelial cell monolayer, are injured by placing stents or other devices/implants into a vessel.

Moreover, the present invention refers to a pharmaceutical composition comprising an extract of Arthrospira, in particular Spirulina and/or phycobiliproteins for use in the prevention or treatment of a blood vessel disease selected from the group of neo-intimal hyperplasia, restenosis, thrombosis or embolism, wherein a medical device for implantation into a bodily vessel or luminal structure is coated with an extract of Arthrospira, in particular Spirulina.

According to the present invention such a said blood vessel injury may result in further complications like bacterial infections, bloodstream infections, primary bacteremia and sepsis. As outlined in the examples and figures an extract of Arthrospira, in particular Spirulina is effective against bacterial toxins like lipopolysaccharides.

In particular, the present invention refers to a pharmaceutical composition comprising an extract of Arthrospira, in particular Spirulina and/or phycobiliproteins for use in the prevention or treatment of bacterial infections, preferably bloodstream infections, primary bacteremia and sepsis.

Moreover, the present invention refers to a pharmaceutical composition comprising an extract of Arthrospira, in particular Spirulina and/or phycobiliproteins for use in the prevention or treatment of a disease selected from the group of bacterial infections, preferably bloodstream infections, primary bacteremia and sepsis, wherein a medical device for implantation into a bodily vessel or luminal structure is coated with an extract of Arthrospira, in particular Spirulina and/or phycobiliproteins.

Moreover, the present invention refers to a medical device for implantation into a bodily vessel or luminal structure as outlined above, wherein said medical device has a coating and the coating comprises an extract of Arthrospira or Spirulina and/or phycobiliproteins, and wherein the coating is for use in the prevention or treatment of a disease selected from the group of blood vessel disease selected from the group of neo-intimal hyperplasia, restenosis, thrombosis or embolism or for use in the prevention or treatment of a disease selected from the group of bacterial infections, bloodstream infections, primary bacteremia and sepsis.

All mentioned diseases and defects are well described in Pschyrembel (e.g. https://www.pschyrembel.de/).

The active agents, i.e. an extract of Arthrospira, in particular Spirulina and/or phycobiliproteins may be administered by conventional methods for solid drug preparations mixing e.g. all active agents and pelletizing them for example into pellets together with conventional excipients or auxiliary materials.

The pharmaceutical preparations may be administered in liquid or solid form for oral, enteral or parenteral application including intravenous routes. In this connection all conventional forms of application are possible, in particular it is available in a galenic formulation like tablets, coated tablets, pellets, capsules, dragées, sirups, solutions, suspensions. Preferably, water is used as an injection medium containing added substances common in injection solutions such as stabilizers, dissolving intermediaries and buffers. If desired, preparations suited for oral application may contain flavorings or sweeteners.

As used herein the terms “treating” and “treatment” or “preventing” and “prevention” refer to any and all uses which remedy a condition or symptoms, prevent the establishment of a condition or disease, or otherwise prevent, hinder, retard, or reverse the progression of a condition or disease or other undesirable symptoms in any way whatsoever. Thus, the terms “treating” and “treatment” or “preventing” and “prevention” are to be considered in their broadest context. For example, treatment does not necessarily imply that a subject is treated until total recovery.

As used herein the term “subject” includes humans and animals. Typically, the subject is a human, or a patient.

In the following, the present invention is described in more detail by way of examples and figures. However, these examples are not intended to limit the scope of protection of the present invention in any way.

### EXAMPLES

### Example 1: Protocol for Cell Index (CI) Measurement by RT-CES System

The xCELLigence system is available from ACEA Biosciences, San Diego, CA, USA: It consists of a plate station with up to six 96 well E-Plates and a software for automatic and real-time data acquisition and display. The system measures electrical impedance across micro-electrodes integrated on the bottom of tissue culture E-Plates. It allows to monitor changes of adherence, spreading and proliferation of HUVEC (human umbilical vascular endothelial cells) or other cells in real time based on the measured cell-electrode impedance. From these data, a parameter termed “Cell Index (CI)” can be calculated, according to

$$CI = \begin{matrix}max \\ i = 1 , .. , N\end{matrix} [ ( \frac{Rcell ( fi )}{Rb ( fi )} - 1 ) ]$$

where R_{b}(f) and R_{cell}(f) are the frequency dependent electrode resistances (a component of the impedance) without cells or with cells present, respectively. N is the number of the frequency points at which the impedance is measured.

Thus, CI is a quantitative measure of the status of the cells in an electrode-containing well. Under the same physiological conditions, more cells attached on to the electrodes leads to larger R_{cell}(f) value, leading to a larger value for CI. Furthermore, for the same number of cells present in the well, a change in the morphology of the cells (spread cells) will also lead to a change in the CI. A “Normalized Cell Index” at a given time point is calculated by dividing the Cell Index at the time point by the Cell Index at a reference time point. Thus, the normalized Cell Index is 1 at the reference time point.

In the absence of living cells (cell culture medium only) or with a suspension of dead cells, the cell index values will be close to zero. After cellular attachment onto the electrode, the measured signal correlates linearly with cell number throughout the experiment with sufficient accuracy, which has been shown in many publications [see e.g. 15].

### Example 2: Cultivation of HUVEC and Preparation of SP Extract

### Endothelial Cells

Human vein endothelial cells (HUVEC) used in this study were purchased from Lonza (Basel, Switzerland). HUVEC were cultured in a standard humidified incubator at 37° C. with 5% CO₂ according to optimal media and growth conditions. Cells were used at passage 4 for experiments.

### Preparation Spirulina Extract (in Short: SP) for Cell Culture Experiments

Spirulina powder (BioSpirulina, Sanatur GmbH, Singen, Germany) was stirred overnight in sterile 0.9% NaCl solution (B. Braun, Melsungen, Germany) at room temperature (10 mg/ml). Then the extract was centrifuged at 3400 g for 5 minutes with subsequent filtration using a 0.45 and 0.22 μm filter. The extract was stored at 4° C. until further processing.

### Example 3: Influence of SP on HUVEC Monolayer Formation and its Influence on Lipopolysaccharides (in Short: LPS)-Mediated Cytotoxicity

### Study Design

The experiments were designed as prospective, controlled in vitro study using human umbilical vein endothelial cells (HUVEC). Optimal HUVEC number was determined in a set of experiments in order to obtain a significant cell index value and a constant cell growth during the entire duration of the experiment. From these experiments the optimum cell density was chosen as 3000 cells/well.

### Influence of SP on HUVEC Monolayer Formation

Thereafter, the intrinsic effect of SP in increasing concentrations (0.125 μg/ml, 50 μg/ml, 250 μg/ml) on viability, adhesion and proliferation of human vein endothelial cells was assessed in real-time by the xCELLigence system E-plate 16 (ACEA Biosciences, Inc.) for 80 h at 10 min intervals.

Depending on the concentration of SP an increase of the cell index was observed 56 h after adding Spirulina extract (SP). The cell index (CI) after the addition of 0.125 μg/ml Spirulina was CI=4.53, after adding 50 μg/ml CI=5.25 and CI=4.97 for the addition of 250 μg/ml SP.

TABLE 1
Tukey-Kramer significance levels for
Spirulina platensis supplementation.

| | Control | 0.125 μg/ml | 50 μg/ml | 250 μg/ml |
|---|---|---|---|---|
| Control | — | — | 0.05 | 0.05 |
| 0.125 μg/ml | | — | 0.05 | — |
| 50 μg/ml | | | — | — |
| 250 μg/ml | | | | — |

FIG. 2 shows the HUVEC density after supplementation of the cell culture medium with different concentrations of SP in comparison to untreated HUVEC over cultivation time.

### Influence of Lipopolysaccharides (in Short: LPS) on HUVEC Monolayer Formation

By adding LPS of E. coli (5 μg/ml, Merck KGaA, Darmstadt, Germany [product L6638, dissolved in DMSO], hereinafter: LPA) as toxic component of bacterial lipopolysaccharides (LPS), a significant and concentration-dependent decrease of the HUVEC monolayer density was observed. The CI was diminished after the addition of 5 μg/ml LPA to the cell culture medium to 3.61 compared to 4.155 of untreated HUVEC. It became obvious that most of the HUVEC detached immediately after LPA treatment for a short time period (see CI FIG. 3 below the arrow “treatment”), followed by renewed attachment. This effect was not seen for untreated HUVEC or HUVEC treated with SP (FIGS. 1 and 2) although the same volumes (medium or medium with SP extract) were added to the cells. This seemed to be a specific effect of LPA.

The two samples differed significantly (ANOVA: p<0.001). Tukey-Kramer test showed that the control culture differed from the HUVEC supplemented with 5 μg/ml LPA (p<0.05).

TABLE 2
Tukey-Kramer significance levels for LPA
supplementation in [μg/ml].

| | Control | 5 μg/ml LPA |
|---|---|---|
| Control | — | 0.05 |

FIG. 3 shows the HUVEC density after supplementation of the cell culture medium with 5 μg/ml LPA in comparison to untreated HUVEC over cultivation time.

Effect of SP on Endothelial Cell Impairment by LPA (5 μg/Ml)

The addition of 5 μg/ml LPA to the cell culture medium (red curve) induced—as already seen in FIG. 3—a substantial decrease of adherent HUVEC compared to control cells from 4.155 to 3.61 56 hours after adding LPA. This decline was completely compensated by the initial addition of 0.125 μg/ml SP. A harmful effect of LPA on HUVEC was not observed anymore.

After the supplementation of the cell culture medium with 50 μg/ml SP and then adding 5 μg/ml LPA even more HUVEC adhered/proliferated compared to untreated control cells (FIG. 4). So in this case, a suitable concentration of SP even overcompensated the toxic effect of LPA.

TABLE 3
Tukey-Kramer significance levels for LPA supplementation.

| | | | 5 μg/ml | 5 μg/ml |
|---|---|---|---|---|
| | | 5 μg/ml | LPA + | LPA + |
| | Control | LPA | 0.125 μg/ml SP | 50 μg/ml SP |
| Control | — | 0.05 | — | — |
| 5 μg/ml LPA | | — | — | 0.05 |
| 5 μg/ml | | | — | 0.05 |
| LPA + 0.125 μg SP | | | | — |
| 5 μg/ml LPA + 50 μg | | | | |
| SP | | | | |

FIG. 4 shows the development of HUVEC densities over time after supplementation of the cell culture medium with 5 μg/ml LPA in absence or presence of different SP concentrations.

The study revealed that the aqueous extract of Spirulina i.) led—in comparison to control cells—to an accelerated formation of an endothelial cell monolayer and ii.) had a protective effect against endothelial cell detachment due to LPS and depending on the SP-concentration of the extract. The harmful effect of LPA could be reversed by a concentration of 0.125 μg/ml Spirulina, and at a concentration of 50 μg/ml there was an accelerated endothelialization despite LPA.

### Example 4

Cultivation of primary human endothelial cells (HUVEC) and preparation of AP extract (“Spirulina” extract) were done as described in Example 2.

HUVEC from the same commercially available lot (Lonza, Basel, Switzerland) were used in both series of experiments. In each case, 1.5*10⁴ cells/ml per well were seeded into a 24 well cell culture plate. Cells were cultured in endothelial cell growth medium-2 (EGM2, Lonza). Settling took place on day 0, the first medium change on day 2, the second medium change on day 4. In each case, before the medium change, the number of adherents HUVEC was quantified.

Spirulina extract (AP) was prepared in isotonic NaCl solution as described in Example 2. Commercially available lyophilized Phycobiliproteins (PC) from Spirulina (Sigma-Aldrich by Merck KGaA, Darmstadt, Germany) was dissolved in phosphate buffered saline (PBS). The volumes added to the cell culture media are summarized in Table 4:

TABLE 4
Additions to cell culture media

| | | |
|---|---|---|
| PC 1.5 [μg/ml] | 5 ml EGM2 + 15 μl PC in PBS | 0.3% of culture medium |
| PC 3.0 [μg/ml] | 5 ml EGM2 + 30 μl PC in PBS | 0.6% of culture medium |
| PC 6.0 [μg/ml] | 5 ml EGM2 + 60 μl PC in PBS | 1.2% of culture medium |
| AP 50 [μg/ml] | 6.06 ml EGM2 + 15 μl AP extract | 0.25% of culture medium |
| AP 100 [μg/ml] | 6.06 ml EGM2 + 30 μl AP extrakt | 0.5% of culture medium s |
| AP 200 [μg/ml] | 6.06 ml EGM2 + 60 μl AP extrakt | 1.0% of culture medium |

### Results:

Table 5 shows HUVEC densities [cells/cm²] 2 and 4 days after the addition of Arthrospira extract at three concentrations.

Clearly, the proliferation of HUVEC after 100 μg/ml is the strongest with 180% both compared to the control cells (130%) and compared to the other two AP concentrations (50 μg/ml: 161.6%; 200 μg/ml: 145.8%).

TABLE 5
HUVEC densities in [cells/cm2] 2 and 4 days after the
addition of Arthrospira extract at three concentrations.

| | | | Comparison to baseline | |
|---|---|---|---|---|
| | | | absolute | % increase |
| Conditions | day 2 | day 4 | day 2 to day 4 | day 2 to day 4 |
| Untreated control | 20273 ± 3376 | 46720 ± 6474 | 26447 | +130.5 |
| AP 50 [μg/ml] | 23861 ± 2351 | 62428 ± 4036 | 38567 | +161.6 |
| AP 100 [μg/ml] | 25332 ± 3205 | 70931 ± 4850 | 45599 | +180.0 |
| AP 200 [μg/ml] | 19736 ± 4294 | 48520 ± 7349 | 28784 | +145.8 |

Table 6 shows HUVEC densities 2 and 4 days after the addition of phycocyanin at three concentrations.

TABLE 6
HUVEC densities in [cells/cm2] 2 and 4 days after
the addition of phycocyanin at three concentrations.

| | | | Comparison to baseline | |
|---|---|---|---|---|
| | | | absolute | % increase |
| Conditions | day 2 | day 4 | day 2 to day 4 | day 2 to day 4 |
| Untreated control | 21395 ± 1653 | 61080 ± 8573 | 39685 | +185.5 |
| PC 1.5 [μg/ml] | 20744 ± 7737 | 54545 ± 9001 | 33801 | +162.9 |
| PC 3 [μg/ml] | 14859 ± 1912 | 87665 ± 13041 | 72806 | +489.9 |
| PC 6 [μg/ml] | 16415 ± 2327 | 49348 ± 11398 | 32933 | +200.6 |

Again, for the mean concentration of phycocyanin (PC 3 [μg/ml])—which is approximately the same as the concentration of phycocyanin in the 100 μg/ml Arthrospira extract (AP)—there is a significant increase in the proliferation of adherent endothelial cells compared to the control cells and the cultures with addition of 1.5 μg/ml and 6 μg/ml phycocyanin, respectively.

With regard to the results, it should be taken into account that, in addition to the slightly different degree of dilution of the cell culture medium, as well as the different solution media, the different purity of phycocyanin is of particular importance. The purity of commercially available phycocyanin (Sigma) was 3.7, whereas the purity of our extract is 1.

These experiments clearly show that not only aqueous AP extracts increase the growth rate of human endothelial cells and protect against bacterial noxes which is of therapeutic benefit. Also purified ingredients such as phycocyanin increase the growth rate of endothelial cells and promote endothelialization.

Thus, application of purified AP ingredients such phycocyanin, xanthophyll, chlorophyll, beta-carotene, echinenone, xanthine, fatty acids, linolenic acid (ALA), linoleic acid, stearidonic acid (SDA), eicosapentaenoic acid (EPA), docosahexaenoic acid (DHA), arachidonic acid (AA), oligosaccharides, and polyphenols provided in a pharmaceutical composition can be used for
Coated implants to improve endothelialization and to protect against bacterial noxes to prevent thrombosis and embolia
As a pill or infusion to improve endothelialization of an implant or as a systemic medicament to prevent thrombosis and embolia and protect against bacterial noxes

### Example 5

Arthrospira platensis (AP) and some of its derived products have well-established biological activities as antioxidants or as agents to reduce cardiovascular disease risk factors. Furthermore, AP products have gained increasing importance as potential anti-cancer agents. However, the ingredients of the available products vary greatly with the origin, the type of production and processing, which could have significant consequences for their biological effects. Therefore, the composition and biological influence of five distinct AP powders, which were acquired commercially or produced at a public biotechnology institute, were investigated in regard to their endothelialization capacity using a cell impedance- (CI) based measurement method.

### Study Design

The experiments were designed as controlled in vitro study using human umbilical vein endothelial cells (HUVEC). Optimal HUVEC number of 3500 cells/well was determined in a set of experiments in order to obtain a significant cell index value and a constant cell growth during the entire duration of the experiment.

Thereafter, the effect of AP in increasing concentrations (50 μg/ml, 100 μg/ml, 200 μg/ml) on viability, adhesion and proliferation of human vein endothelial cells was assessed in real-time by the xCELLigence system E-plate 16 (see Example 1) from ACEA Biosciences, Inc. for more than 90 h at 30 min intervals.

### Endothelial Cells

HUVEC used in this study were purchased from Lonza (Basel, Switzerland). The cells were cultured in a standard humidified incubator at 37° C. with 5% CO₂ according to optimal media and growth conditions. HUVEC were used at passage 4 for experiments.

### Preparation Arthrospira Extract for Cell Culture Experiments

AP powder were purchased by four providers (A: BioSpirulina-Taiwan (Spirulina platensis), Sanatur GmbH, Singen, Germany; B: Premium Spirulina (Spirulina platensis), Aspermühle, Goch-Asperden, Germany; C: Bio Spirulina (Spirulina platensis), Aspermühle, Goch-Asperden, Germany; D: Ivarssons Hawaian Spirulina (Spirulina pacifica), Schriesheim, Germany;), the fifth powder (E) (Spirulina platensis) was produced at the Institute of Biotechnology, Brandenburg University of Technology, Senftenberg, Germany). Powder E was produced in a vertical flat-type bioreactor of 1.7 L working volume using transparent polyethylene food-safe bags. The AP growth was followed continuously by monitoring optical density and intermittently by measuring the dry weight of the AP biomass. The oxygen produced by AP in the culture medium was flushed out sparging using a mixture of air and CO₂ (1%). Factors which might influence the AP growth were monitored: pH, temperature, oxygen concentration and the filling level were corrected automatically to compensate evaporation losses. The bioreactor was thermostated to 25° C. and externally illuminated by LED lamps with adaptable photon flux densities from 0 to 5000 μmol/(m²·s) applied at a photoperiod 24/0 h. Further details are described in the literature (Jung C G H, Waldeck P, Petrick I, Braune S, Küpper J-H, Jung F. Bioreactor for the cultivation of Arthrospira platensis under controlled conditions. J Cell Biotechnol. 2021. DOI: 10.3233/JCB-210032).

The AP powders were stirred overnight in sterile 0.9% NaCl solution (B. Braun, Melsungen, Germany) at room temperature (10 mg/ml). Then the extracts were centrifuged at 3400 g for 5 minutes with subsequent filtration using a 0.45 and 0.22 μm filter (TPP). The extracts were stored at 4° C. until further processing.

### Results

FIGS. 5a, 6a and 7a show the maximum values of the Cellular Index (CI) representing the proliferation of the seeded endothelial cells after 90 h of cultivation time with supplementation of the culture medium with 50 μg/ml, 100 μg/ml or 200 μg/ml with the five different AP powders. FIGS. 5b, 6b & 7b represents the course of the CI during the whole time of investigation after exposure to the respective AP concentrations.

In Table 7 the pairwise comparison of the CI means of all preparations after 90 h are shown. It can be concluded that preparation D is superior to preparation A, B, C, E, while preparation A is inferior to all preparations.

TABLE 7
Tukey-Kramer test for all pairwise comparisons

| | | | | Different (p < 0.05) |
|---|---|---|---|---|
| Factor | n | Mean | SD | from factor nr |
| (1) A | 8 | 2.7275 | 0.2835 | (B)(C)(D)(E) |
| (2) B | 8 | 3.5225 | 0.2137 | (A)(D |
| (3) C | 8 | 3.7587 | 0.5246 | (A)(D) |
| (4) D | 8 | 4.4588 | 0.1900 | (A)(B)(C)(E) |

The addition of the five AP preparations to the cell culture medium affected the growth of HUVEC significantly (p<0.001). The lowest HUVEC growth was observed after the addition of 100 μg/ml of preparation A with 2.33±0.93, while the highest increase was observed for dry powder D with 4.87±0.2. This corresponds with an increase of 109%. FIG. 6b shows the increase of the CI after supplementation with the different powder.

In Table 8 the pairwise comparisons of the CI means of all preparations after 90 h are presented. While dry powder D is superior to all preparations, preparation A is inferior to preparation C, D and E.

TABLE 8
Tukey-Kramer test for all pairwise comparisons (p < 0.05)

| | | | | Different (p < 0.05) |
|---|---|---|---|---|
| Factor | n | Mean | SD | from factor nr |
| (1) A | 8 | 2.3312 | 0.9393 | (C)(D)(E) |
| (2) B | 8 | 3.1300 | 0.1828 | (D) |
| (3) C | 8 | 3.6687 | 0.4708 | (A)(D) |
| (4) D | 8 | 4.8662 | 0.2033 | (A)(B)(D)(E) |
| (5) E | 8 | 3.7187 | 0.7634 | (A)(D) |

The addition of the five AP preparations to the cell culture medium affected the growth of HUVEC significantly (p<0.001). The lowest HUVEC increase occurred after the addition of 200 μg/ml powder A with 2.26±0.46, while the highest growth was seen after adding powder D with 4.78±0.53 corresponding with an increase on 211.5%. FIG. 7b shows the continuous increase of the CI after supplementation with the different powder.

In Table 9 the pairwise comparisons of the CI means of all preparations after 90 h are presented. While preparation D is superior to all other preparations, power A is inferior to C, D and E.

TABLE 9
Tukey-Kramer test for all pairwise comparisons

| | | | | Different (p < 0.05) |
|---|---|---|---|---|
| Factor | n | Mean | SD | from factor nr |
| (1) A | 8 | 2.2625 | 0.4599 | (B)(C)(D)(E) |
| (2) B | 8 | 3.2125 | 0.4018 | (A)(D) |
| (3) C | 8 | 3.2762 | 0.3927 | (A)(D) |
| (4) D | 8 | 4.7787 | 0.5271 | (A)(B)(C)(E) |
| (5) E | 8 | 3.6675 | 0.3967 | (A)(D) |

The study revealed that the AP composition and especially the influence on HUVEC proliferation differed significantly between the five AP powders up to 109%.

Thus, a method is provided that allows the reliable detection of quantitative differences in biological effects of different AP preparations.

The following concentrations are indicated for

Phycocyanin: (μg/ml)
A=0.81±0.03
B=1.67±0.009
C=1.59±0.006
D=0.70±0.01
E=1.69±0.125
   Allphycocyanin: (μg/ml)
A=0.40±0.005
B=0.61±0.003
C=0.50±0.001
D=0.53±0.008
E=0.39±0.045
   Phycoerythrin: (μg/ml)
A=0.11±0.0001
B=0.19±0.0002
C=0.14±0.0001
D=0.16±0.003
E=0.12±0.045

### FIGURES

FIG. 1: Sketch of measurements of the Cellular Index under different conditions over time.

FIG. 2: Course of mean normalized Cell Index curves during the cultivation of control HUVEC), after adding 0.125 μg/ml SP, 50 μg/ml SP, 250 μg/ml SP or 1000 μg/ml SP (symbols for each defined in graph). Presented are graphs of arithmetic means for n=7 experiments (each treatment).

FIG. 3: Mean normalized Cell Index curves of untreated HUVEC monolayer formation in cell culture wells compared to LPA-treated HUVEC during the cultivation time of 80 hours. Presented are graphs of arithmetic means of n=7 experiments per treatment

FIG. 4: Effect of SP on LPA-induced (5 μg/ml) HUVEC impairment/-detachment compared to control cells during the cultivation time of 80 hours. Mean normalized CI of control cultures, CI of HUVEC treated with 5 μg/ml LPA, CI of HUVEC treated with 0.125 μg/ml SP+5 μg/ml LPA, CI of HUVEC treated with 50 μg/ml SP+5 μg/ml (curve symbols defined in graph). Presented are graphs of arithmetic means of n=7 experiments per treatment.

FIG. 5: Maximum values (FIG. 5a) and course of the CI (FIG. 5b) 90 h after supplementing the culture medium with 50 [μg/ml] of five different Arthrospira platensis powder.

FIG. 6: Maximum values (FIG. 6a) and course (FIG. 6b) of the CI 90 h after supplementing the culture medium with 100 [μg/ml] of five different Arthrospira platensis powder.

FIG. 7: Maximum values (FIG. 7a) and course (FIG. 7b) of the CI 90 h after supplementing the culture medium with 200 [μg/ml] of five different Arthrospira platensis powder.

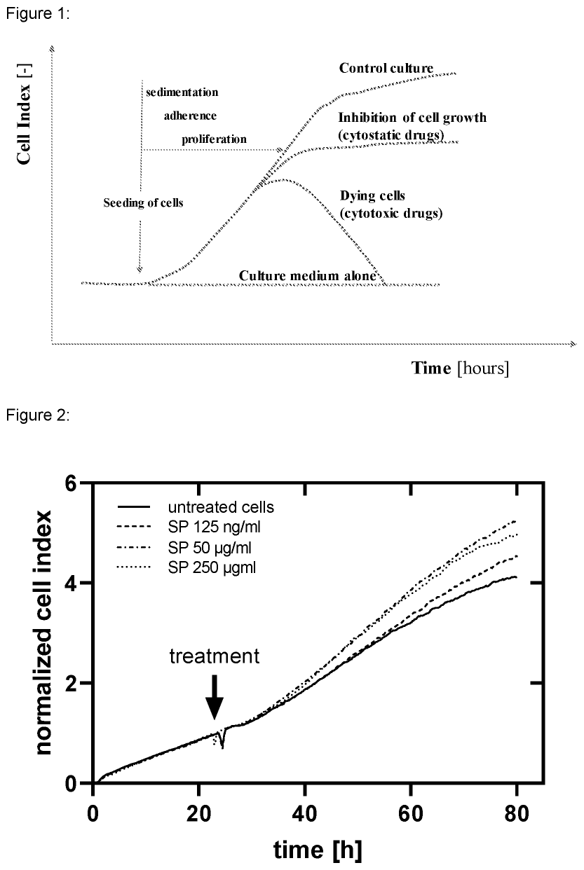

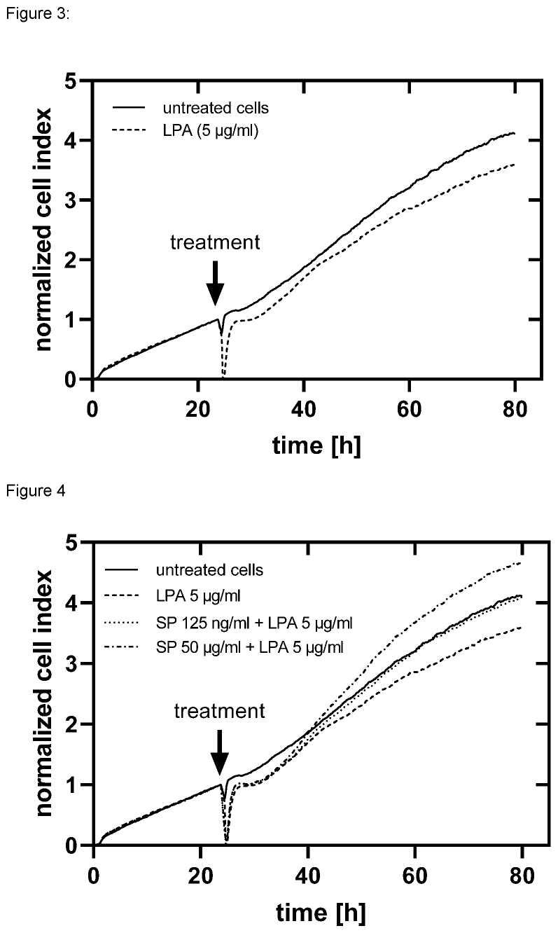

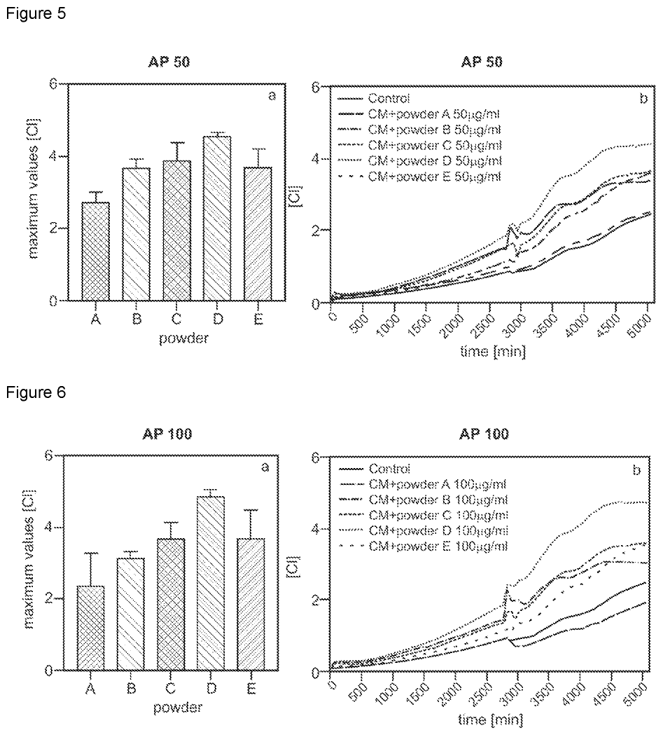

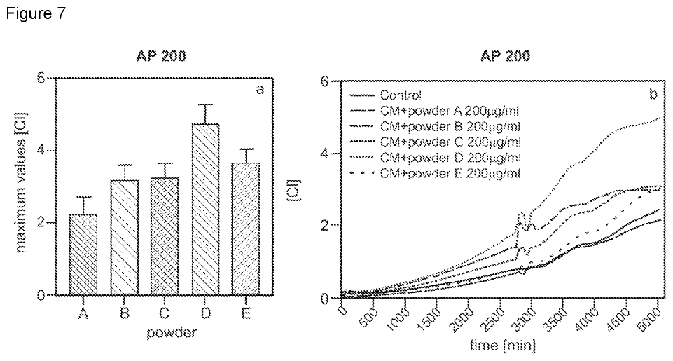

### REFERENCES

[1] Jung F, Krüger-Genge A, Waldeck P, J-H. Küpper J-H. Spirulina platensis, a super food? J Cell Biotechnol. 2019; 5:43-54.
[2] Khan Z, Bhadouria P, Bisen P S. Nutritional and therapeutic potential of Spirulina. Curr Pharm Biotechnol. 2005; 6:373-379.
[3] Miczke A, Szulinska M, Hansdorfer-Korzon R, Kregielska-Narozna M, Suliburska J, Walkowiak J, Bogdanski P. Effects of Spirulina consumption on body weight, blood pressure, and endothelial function in overweight hypertensive Caucasians: a double-blind, placebo-controlled, randomized trial. European Review for Medical and Pharmacological Sciences. 2016; 20: 150-6.
[4] Kuhad A, Tirkey N, Pilkhwal S, Chopra K. 2006a. Renoprotective effect of Spirulina fusiformis on cisplatin-induced oxidative stress and renal dysfunction in rats. Ren Fail. 2006 28: 247-254. 2016; 20:150-156.
[5] Dartsch P C. Antioxidant Potential of Selected Spirulina platensis Preparations. Phytother Res. 2008; 22, 627-633.
[6] Chu W-L, Lim Y-W, Radhakrishnan A K, Lim P E. Protective effect of aqueous extract from Spirulina platensis against cell death induced by free radicals. BMC Complement Altern Med. 2010; 10:53.
[7] Bhat V B, Madyastha K M. Scavenging of peroxynitrite by phycocyanin and phycocyanobilin from Spirulina platensis: protection against oxidative damage to DNA. Biochem Biophys Res Commun. 2001; 285:262-266.
[8] Pinero Estrada J E, Bermejo Bescos P, Villar del Fresno A M. Antioxidant activity of different fractions of Spirulina platensis protean extract. Farmaco. 2001; 56:497-500.
[9] Heo S-Y, Ko S C, Kim C S, Oh G-W, Ryu B, Qian Z-J, Kim G, Park W S, Choi W-I, Phan T T V, Heo S-J, Kang D-H, Yi M, Jung W-K. A heptameric peptide purified from Spirulina sp. gastrointestinal hydrolysate inhibits angiotensin I-converting enzyme- and angiotensin II-induced vascular dysfunction in human endothelial cells. International Journal of Molecular Medicine. 2017; 39:1072-1082.
[10] Viswanadha V P, Sivan S, Rajendra Shenoi R. (2011). Protective effect of Spirulina against 4-nitroquinoline-1-oxide induced toxicity. Mol Biol Rep 38:309-17.
[11] Patil et al., Method to obtain C-phycocyanin of high purity. Journal of Chromatography A, 1127 (2006) 76-81.
[12] Kumar et al., Extraction and purification of C-phycocyanin from Spirulina platensis (CCC540), Ind J Plant Physiol. 19(2):184-188.
[13] Braune, S. Krüger-Genge, A. Kammerer, S. Jung, F., Küpper, J.-H. Phycobiliproteins from Arthrospira platensis as potential anti-cancer drug: review of in vitro and in vivo studies. Life, 11, 91. https://doi.org/10.3390/life11020091 2021
[14] Pan-utai, W., Kahapana, W. & Iamtham, S. Extraction of C-phycocyanin from Arthrospira (Spirulina) and its thermal stability with citric acid J Appl Phycol (2018) 30:231-242
[15] Kho D, MacDonald C, Johnson R, Unsworth C P, Carroll S J, Mez E D, Angel C E, Graham E S. Application of xCELLigence RTCA Biosensor Technology for Revealing the Profile and Window of Drug Responsiveness in Real Time. 2015, 5, 199-222.

1.-14. (canceled)

15. Pharmaceutical composition comprising an extract of Arthrospira or Spirulina and/or phycobiliproteins for use in the prevention or treatment of a blood vessel disease selected from the group of neo-intimal hyperplasia, restenosis, or embolism.

16. Pharmaceutical composition comprising Arthrospira or Spirulina and/or phycobiliproteins for use in the prevention or treatment of a disease selected from the group consisting of bloodstream infections, primary bacteremia and sepsis.

17. Pharmaceutical composition comprising an extract of Arthrospira or Spirulina for use in the prevention or treatment of a disease according to claim 15, wherein the extract is an enriched extract of Arthrospira or Spirulina which comprises more than 15 w/w phycobiliproteins.

18. Pharmaceutical composition comprising an extract of Arthrospira or Spirulina and/or phycobiliproteins for use in the prevention or treatment of a disease according to claim 15, wherein a medical device for implantation into a bodily vessel or luminal structure is coated with an extract of Arthrospira or Spirulina and/or phycobiliproteins.

19. Pharmaceutical composition for use in accordance with claim 15, wherein the phycobiliproteins are selected from the group consisting of phycocyanin, C-phycocyanin, allophycocyanin, R-phycocyanin and phycoerythrin.

20. A medical device for implantation into a bodily vessel or luminal structure, wherein said medical device has a coating and the coating comprises an extract of Arthrospira or Spirulina and/or phycobiliproteins, wherein the implantable device is selected from the group consisting of stents, stent-grafts, synthetic bypass grafts, embolic filters, occluder systems, detachable coils, pacemaker and defibrillator leads, plates, screws, spinal cages, dental implants, ventricular assist devices, artificial hearts, artificial heart valves, annuloplasty devices, artificial joints, and implantable sensors.

21. A medical device for implantation into a bodily vessel or luminal structure according to claim 22, wherein the coating is for use in the prevention or treatment of a disease selected from the group of blood vessel disease selected from the group of neo-intimal hyperplasia, restenosis, or embolism or for use in the prevention or treatment of a disease selected from the group of bloodstream infections, primary bacteremia and sepsis.

22. Pharmaceutical composition for use in accordance with claim 15, characterized in that it is available in a galenic formulation.

23. Pharmaceutical composition for use or a medical device in accordance with claim 15, wherein Arthrospira is selected from the species of A. platensis, A. maxima and A. fusiformis.

24. Pharmaceutical composition for use or a medical device in accordance with claim 15, wherein an extract of Arthrospira or Spirulina is enriched with bioactive compounds by means of concentration, fractionation, or addition.

25. Pharmaceutical composition or a medical device in accordance with claim 15, wherein an extract of Arthrospira or Spirulina is enriched with compounds selected from the group of secondary plant substances, photosynthetically active pigments, phycobiliproteins, phycocyanin, xanthophyll, chlorophyll, beta-carotene, echinenone, xanthine, fatty acids, linolenic acid (ALA), linoleic acid, stearidonic acid (SDA), eicosapentaenoic acid (EPA), docosahexaenoic acid (DHA), arachidonic acid (AA), oligosaccharides, polyphenols.
